(19) United States
(12) Patent Application Publication
(10) Pub. No.: US 2021/0252176 A1
(43) Pub. Date: Aug. 19, 2021

(54) AIR SHOWER GATE

(71) Applicants: Dong Kyu NAM, Seongnam-si, Gyeonggi-do (KR); PURIUM CO., LTD., Siheung-si, Gyeonggi-do (KR)

(72) Inventor: Dong Kyu NAM, Seongnam-si (KR)

(21) Appl. No.: 17/059,079

(22) PCT Filed: Jul. 2, 2019
(86) PCT No.: PCT/KR2019/007996

§ 371 Date: Nov. 25, 2020

(30) Foreign Application Priority Data
Jul. 4, 2018 (KR) 10-2018-0077655

(51) Int. Cl. A61L 2/00; F24F 9/00; B08B 5/02; A61L 2/24

(52) U.S. Cl. CPC A61L 2/0005; F24F 9/00; F24F 2120/10; A61L 2/24; B08B 5/02

(57) ABSTRACT

The present invention relates to an air shower gate and, more specifically, to an air shower gate installed at an indoor/outdoor entrance/exit so as to remove microdust, hazardous materials, and odor from people entering/exiting same, and configured to monitor the quality of indoor/outdoor air such that, through air purification or ventilation, a comfortable and clean indoor environment can be maintained and managed. An air shower gate according to the present invention has the following advantages: Dust and the like gathered on clothes during outdoor activities can be removed, and indoor and outdoor spaces are separated by an air curtain, thereby enabling the user to move in/out conveniently without having to open/close a door. Inflow of dust into the indoor space is prevented by the air curtain, and indoor air is filtered to perform an air purifying function.

### TECHNOLOGY FIELD

The present invention relates to air shower gates, more specifically, to an air shower gate that is to be installed at an entrance between an indoor space and an outdoor space, to remove particulates, hazardous substances and odors from a person passing the entrance, to monitoring indoor/outdoor air quality, and to maintain a comfortable and clean indoor environment by air purification or ventilation.

### TECHNICAL BACKGROUND

In general, air shower devices are used to remove dust from clothes in an air shower booth installed inside an indoor space.

As more people pay attention to particulates in air, various solutions are proposed to enhance indoor air quality.

In schools, apartments, and large buildings, indoor air can be polluted by people entering an indoor space from an outdoor space. Recent increase of air particulates increases demand for a solution to prevent contaminants like dust and dirt from entering indoor spaces.

An air shower device in the form of a conventional air shower booth disclosed in Korean Patent No. 10-1549243 is for indoor installation, and is not suitable for removing contaminants such as dust when people are entering an indoor space.

A conventional air shower booth is for keeping dust away from an industrial facility, and does not provide an appropriate air blowing method or an appropriate air blow strength for an ordinary person. Specifically, air blow from a conventional air shower booth may not be desirable for an ordinary person because it may dishevel hair and blow clothes such as skirts up.

### DETAILED DESCRIPTION OF INVENTION

### Technical Problem

The present invention is to provide an air shower gate that can remove dust collected on clothes from outdoor activities, allow a user to pass conveniently, prevent dust from entering an indoor space, perform air purification by filtering indoor air, provide ventilation by filtering outside air and supply the filtered air into the indoor space, blow air in a desirable manner for a body part of the user, and blow air in a way preferable to the user.

### Solution to Problem

To address the above technical problems, an air shower gate according to an embodiment of the present invention is installed at an entrance of a building between an indoor space and an outdoor space providing a passage for a user. The air shower gate includes two vertical frames installed to neighbor left/right sides of the entrance and further includes a horizontal frame installed to neighbor atop portion of the entrance. The air shower gate includes a first sensor for detecting a human body inside a certain area including the entrance, a second sensor for sensing air quality of the indoor space, a third sensor for sensing air quality of the outdoor space. On an inner side of a vertical frame facing the passage, an operating device, a plurality of side air blow outlets and a plurality of dirty air inlets are provided. On an outer side of the vertical frame, a display device and an outer side air inlet are provided. On an inner side of the horizontal frame facing the passage of the entrance, a plurality of air blow outlets are provided. At an indoor-side edge portion and an outdoor-side edge portion of the horizontal frame, an air curtain is provided respectively.

An inlet for external air is formed on an upper external side of the horizontal frame.

A filter unit for filtering air is provided respectively inside the outer side air inlet and the inlet for external air.

The air shower gate includes a controller that controls the air blow outlets, the air inlets and the air curtains based on sensor information.

The air shower gate may further include, at its vertical frame, a hand sanitizing device that is activated using a proximity sensor.

In addition, the air shower gate can measure a height, a level of shoulder, a range of face location of a user. The control uses the measured data to control air blow outlets.

In addition, an air blow from the side air blow outlet can be inclined downward.

A customized air blow operation can be conducted using preset information received via wireless communication with a user wireless terminal.

Air purification and ventilation can be performed based on air quality information measured using the second sensor and the third sensor.

### Advantageous Effects of Invention

An air shower gate according to an embodiment can remove, from clothes, dust from outdoor activities, and allows a person to pass without opening or closing a door by providing an air curtain that separates an indoor space and an outdoor space. The air shower gate can prevent dust from entering into the indoor space, can perform air purification by filtering indoor air, and provide ventilation by filtering outside air and supply the filtered air into the indoor space according to sensor information. The air shower gate sprays air in a way desirable for a user body part according to sensor information to not dishevel hair, has a direction of side air blow inclined downward to not blow clothes, such as skirts, up and down. The air shower gate can blow air in a way preferable to an individual user using user information received from a wireless terminal such as a smartphone.

### BRIEF DESCRIPTION OF DRAWINGS

FIG. 1 is a perspective view taken from a top, right side according to an embodiment of the present invention.

FIG. 2 is a perspective view from a bottom, left side according to an embodiment of the present invention.

FIG. 3 is a front view presenting an operation according to an embodiment of the present invention.

FIG. 4 is a front view, presenting an operation according to an embodiment of the present invention.

FIG. 5 is a side view presenting an operation according to an embodiment of the present invention.

### BEST MODE FOR OF CARRYING OUT INVENTION

Desirable embodiments of the present invention will be described in detail with reference to the accompanying drawings. The size or shape of elements may be exaggerated in the drawings for clarity and convenience of description. Further, terms particularly defined in consideration of configuration and operation of the present invention are only for describing embodiments of the present invention, and do not limit the scope of the present invention.

It is noted that, regarding reference numerals of elements in the drawings, the same elements have the same reference numerals when possible even they are shown in different drawings. In addition, a detailed description of known configurations and features can be omitted in describing the present invention when such detailed description may obscure an important point of the present invention.

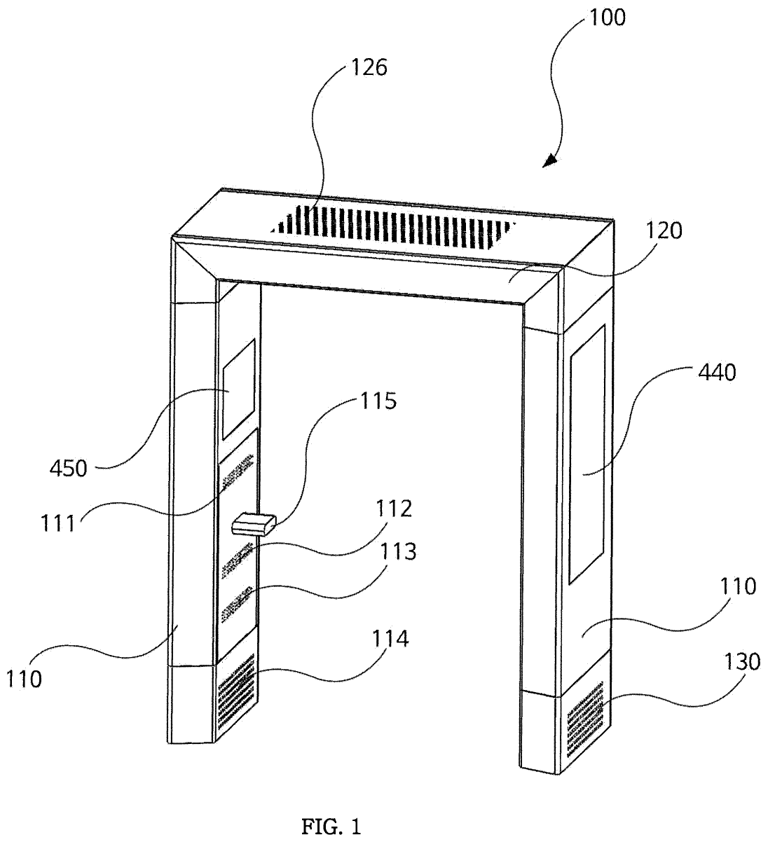

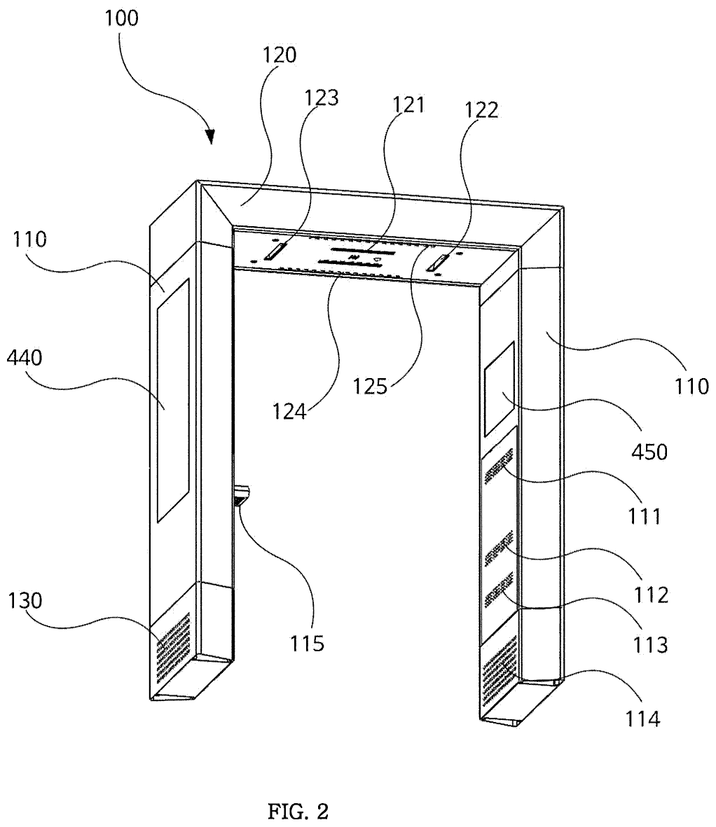

An air shower gate according to an embodiment of the invention will be described below with reference to FIGS. 1-5.

An air shower gate 100 is installed at an entrance between an indoor space and an outdoor space of a building, and is to provide a passage for a user.

The air shower gate 100 according to the present invention includes a vertical frame 110 and a horizontal frame 120.

The air shower gate 100 having the configuration described above can be easily installed to a conventional entrance. The vertical frames 110 are installed on both sides of the entrance and the horizontal frame 120 is installed over the vertical frame 110, which allows the air shower gate to fit various size of entrances. For size adjustment, a horizontal length adjusting member and a vertical length adjusting member can be coupled to a connection part.

The vertical frame 110 is an element installed to abut left and right sides of the entrance. An operating device 450, a first side air blow outlet 111, a second side air blow outlet 112, a third side air blow outlet 113, and a dirty air inlet 114 are provided in order from the top on an inner side of the vertical frame facing the entrance passage. A display device 440 and an outer side inlet 130 are provided on an external side of the vertical frame. In a desirable embodiment of the present invention, each of the air blow outlets 111, 112, 113 blows air independently using a fan with a discharge hole. That allows the controller to individually control turning on/off of air blow and strength of air blow for each of the air blow outlets. The display device 440 is a display that provides various information about air, information about the user, and information that may be necessary inside the building. The display device can receive and display information from an external system.

In addition, air blows from the first side air blow outlet 111, the second side air blow outlet 112 and the third side air blow outlet 113 are inclined downward to not flutter clothes, such as skirts, up and down.

In addition, a hand sanitizing device 115 is provided on an inner side of the vertical frame 110 facing the passage of the entrance. The hand sanitizing device includes a proximity sensor for activating the hand sanitizing device when a hand of the user approaches.

The horizontal frame 120 is a component installed to abut a top portion of the entrance. A first top air blow outlet 121 is formed at a central portion on an inner side of the horizontal frame 120 facing the entrance passage, an second top air blow outlet 122 is formed on one side of the first air blow outlet, and a third top air blow outlet 123 is formed on the other side of the first air blow outlet. A first air curtain 124 is provided on a rear side of the first top air blow outlet close to the indoor space, a second air curtain 125 is provided on a front side of the first top air blow outlet close to the outdoor space, and an external air inlet 126 in provided on an upper side of the horizontal frame. In a desirable embodiment of the present invention, each of the air blow outlets 121,122,123 and the air curtain 124,125 independently blows air using a fan having an air discharge hole. This configuration allows the controller each of the air blow outlets and the air curtains to turn on and off and to adjust air spray strength.

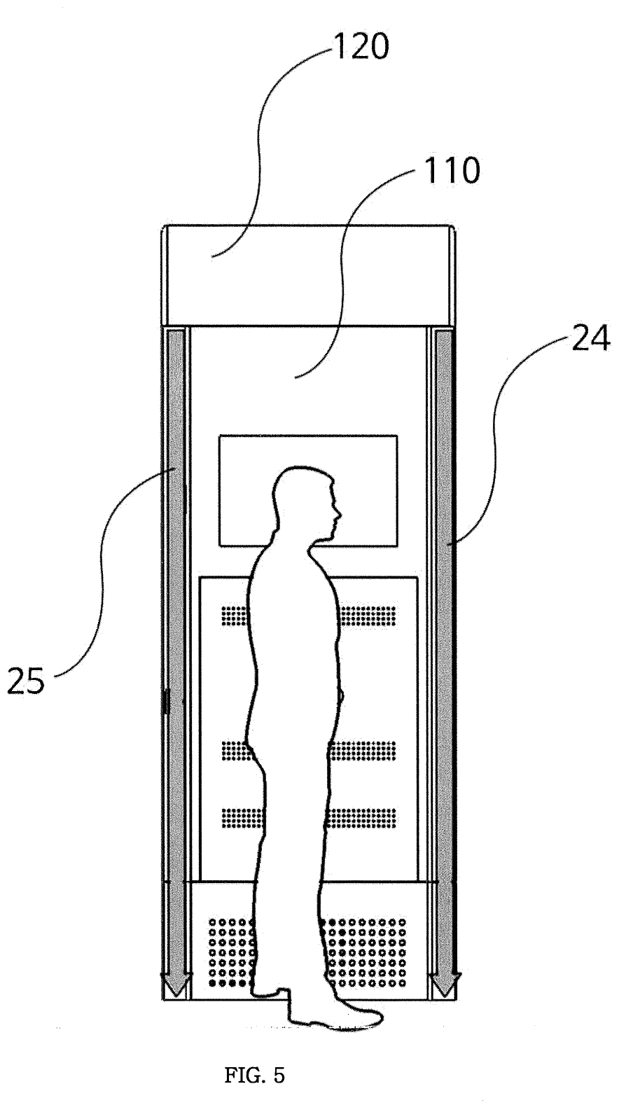

The air curtains 124, 125, when a user is inside a passage space of the air shower gate 100, as shown in FIG. 5, separate the passage space from the indoor space and the outdoor space, and thereby prevent an air flow generated from air dusting of the user to flow outside the passage space and cause the air flow to be drawn into the dirty air inlet entirely.

An embodiment of the present invention includes a first sensor for detecting a human body located at a certain area including the entrance, a second sensor for sensing air quality of the indoor space, a third sensor for sensing air quality of the outdoor space. Each of the sensors can be located at any one of the vertical frames 110 and the horizontal frame 120. In an embodiment of the present invention, the first sensor can recognize a user using an image sensor and further recognize body parts of the user such that the controller controls based on user body information.

In addition, a filter for filtering air is provided inside the outer side inlet 130 and the external air inlet 126 to filter air drawn into the inlets. Filtered air travels inside the frame and is sprayed via air blow outlets.

The controller is an element that controls the first side air blow outlet, the second side air blow outlet, the third side air blow outlet, the dirty air inlet, the first top air blow outlet, the second top air blow outlet, the third top air blow outlet, the first air curtain, and the second air curtain, based on sensing information from the first sensor, sensing information from the second sensor, and sensing information from the third sensor. The controller, based on the sensing information, can turn on and off the air blow outlets individually and also can adjust air spraying strength as well.

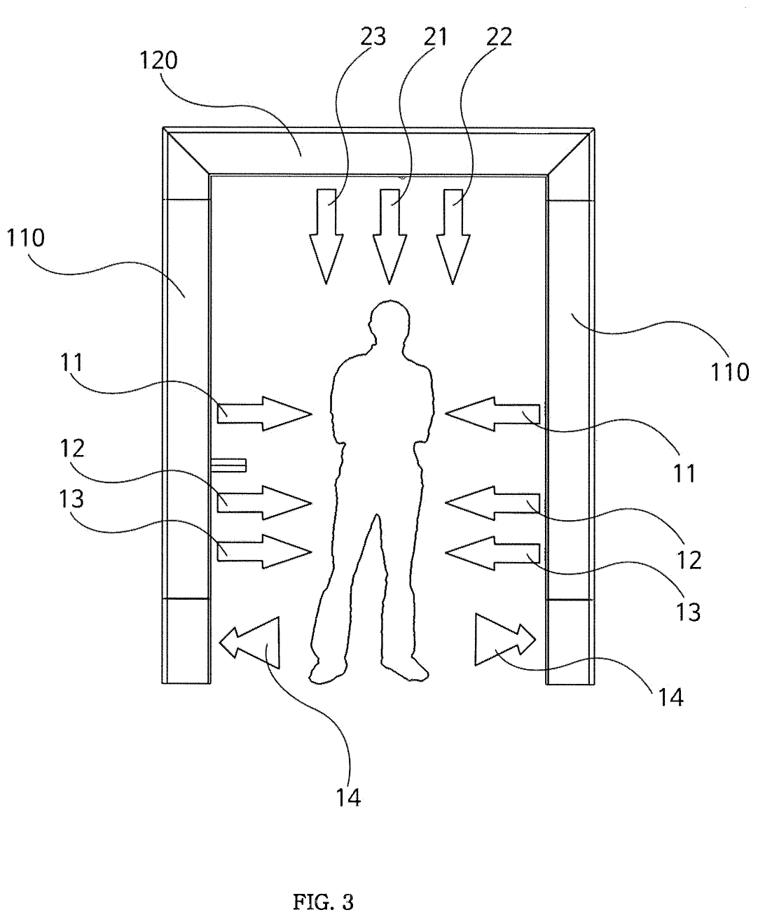

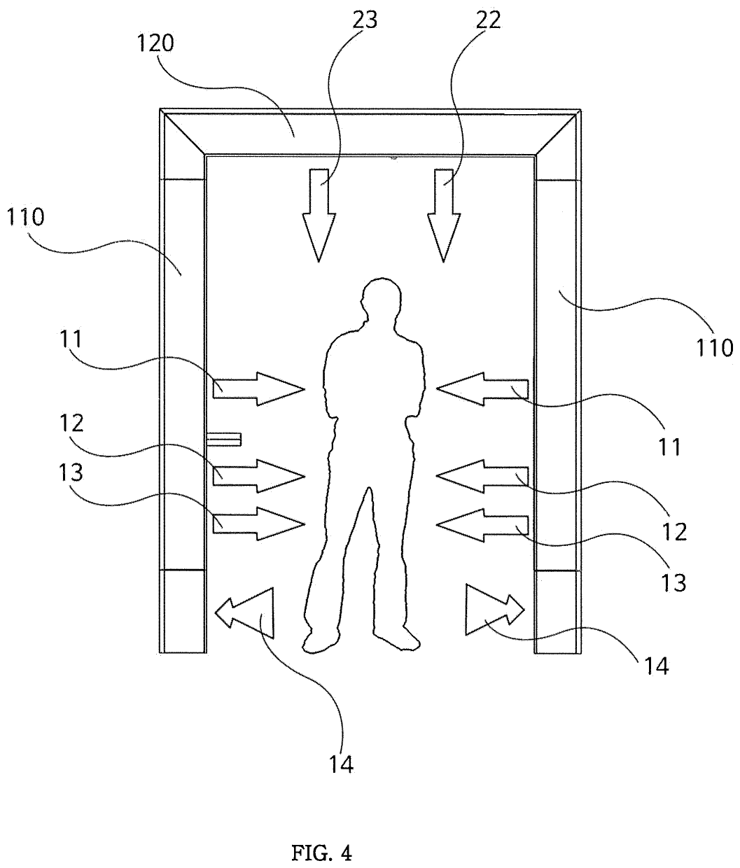

The first sensor measures a height, a level of shoulder, and a range of face location of a user entering the entrance, and include the measure information to sensing information. The controller controls a side air blow outlet located below the shoulder level to spray air, controls a side air blow outlet located above the shoulder level to not spray air, controls an air blow outlet located within the face location range to not spray air, controls an air blow outlet outside the face location range to spray air. FIG. 3 show spraying air to the user's head. FIG. 4 shows the first top air blow outlet 121 not blowing air to the user's head according to a control described above.

In addition, the controller includes a wireless communication transceiver. The wireless communication transceiver receives a preset information including a spraying time period, a spraying intensity, and the like, and enable the controller to control operation of the air blow outlets.

In addition, the second sensor measures a dust density of the indoor space and includes the measured dust density in sensing information, and accordingly, the controller controls the external air inlet and the outer side inlet to take air in when the indoor space dust density is greater than a predetermined dust density and controls an air blow outlet to blow filtered air out.

In addition, the third sensor measures a dust density of the outdoor space and composition ratios of contaminants in air, and includes the measure data in sensing information. When the outdoor dust density and the composition ratios of contaminants are less than a predetermined values, the controller can control the first air curtain and the second air curtain to not operate such that air from the outdoor space flows into the indoor space.

### DESCRIPTION OF REFERENCE NUMERALS

11: first side air blow
12: second side air blow
13: third side air blow
14: dirty air
21: first top air blow
22: second top air blow
23: third top air blow
24: first air curtain blow
25: second air curtain blow
100: air shower gate
110: vertical frame
111: first side air blow outlet
112: second side air blow outlet
113: third side air blow outlet
114: inlet for dirty air
115: hand sanitizing device
120: horizontal frame
121: first top air blow outlet
122: second top air blow outlet
123: third top air blow outlet
124: first air curtain
125: second air curtain
126: external air inlet
130: outer side inlet
440: display device
450: operating device

1-5. (canceled)

6. An air shower gate installed in a building, the air shower gate comprising:
a frame installed between an indoor space of the building and an outdoor space to provide an entrance opening for people to pass therethrough for entering the indoor space of the building from the outdoor space;
at least one sensor configured to detect a person in the entrance opening, a face of the person, a height of the person, and a shoulder level of the person;
at least one controller configured to control an air shower operation of the air shower gate;
the frame comprising a first vertical frame, a second vertical frame, and a top frame;
the first vertical frame extending in a vertical direction;
the second vertical frame extending in the vertical direction and being apart from the first vertical frame in a first horizontal direction;
the top frame extending in the first horizontal direction and connected to the first and second vertical frames;
the top frame extending between an indoor-side edge thereof and an outdoor-side edge thereof in a second horizontal direction perpendicular to the vertical direction and the first horizontal direction;
the top frame comprising an indoor-side air curtain outlet that extends in the first horizontal direction along the indoor-side edge thereof and is configured to generate an indoor-side air curtain that extends from the top frame to a floor in the vertical direction and also extends between the first and second vertical frames in the first horizontal direction;
the top frame comprising an outdoor-side air curtain outlet that extends in the first horizontal direction along the outdoor-side edge thereof and is apart from the indoor-side air curtain in the second horizontal direction, wherein the outdoor-side air curtain outlet is configured to generate an outdoor-side air curtain that extends from the top frame to the floor in the vertical direction, also extends between the first and second vertical frames in the first horizontal direction, and is apart from the indoor-side air curtain in the second horizontal direction;
wherein, when the top frame generates both the indoor-side air curtain and the outdoor-side air curtain, the air shower gate is configured to create an isolated air shower space defined by the frame, the indoor-side air curtain, and the outdoor-side air curtain;
the top frame further comprising a plurality of top air shower outlets configured to generate top air shower blows directed toward the floor, wherein the plurality of top air shower outlets are arranged along the first horizontal direction such that at least one of the plurality of top air shower outlets is located in a central portion of the top frame in the first horizontal direction;
the first vertical frame comprising a plurality of first side air shower outlets configured to generated first lateral air shower blows directed toward the second vertical frame, wherein the plurality of first side air shower outlets are arranged along the vertical direction such that at least one of the plurality of first side air shower outlets is located higher than others of the plurality of first side air shower outlets;
the second vertical frame comprising a plurality of second side air shower outlets configured to generated second lateral air shower blows directed toward the first vertical frame, wherein the plurality of second side air shower outlets are arranged along the vertical direction such that at least one the plurality of second side air shower outlets is located higher than others the plurality of second side air shower outlets;
at least one of the first vertical frame and the second vertical frame comprising at least one inlet configured to take air in;
wherein, in response to detecting a first person in the entrance opening, the air shower gate is configured:
to generate the indoor-side air curtain and the outdoor-side air curtain to create the isolated air shower space separate from the indoor and outdoor spaces with the first person staying inside the isolated air shower space, and
while maintaining the isolated air shower space separate from the indoor and outdoor spaces, to generate the top air shower blows, the first lateral air shower blows and the second lateral air shower blows into the isolated air shower space to dust the first person's body,
wherein, when generating the top air shower blows, based on the at least one sensor's detection of the first person, the controller is configured to selectively inactivate at least one of the plurality of top air shower outlets located in the central portion of the top frame to avoid generating a top air blow that could be directed to the first person's head from the top frame,
wherein, when generating the first lateral air shower blows, based on the at least one sensor's detection of the first person's shoulder level, the controller is configured to selectively inactivate at least one of the plurality of first side air shower outlets to avoid generating a first lateral air blow that could be directed to the first person's head from the first vertical frame,
wherein, when generating the second air shower blows, based on the at least one sensor's detection of the first person's shoulder level, the controller is configured to selectively inactivate at least one of the plurality of second side air shower outlets to avoid generating a second lateral air blow that could be directed to the first person's head from the second vertical frame,
wherein at least one of the plurality of first and second side air shower outlets is configured to generate at least part of the first and second lateral air shower blows in a direction inclined to the floor from the first and second horizontal directions which would avoid lifting of a skirt in case the first person wears the skirt.

7. A method of air showering, the method comprising:
providing an air shower gate that provides an entrance opening of a building for people to pass therethrough to enter an indoor space of the building from an outdoor space;
detecting, by at least one sensor, a person in the entrance opening, a face pf the person, a height of the person, and a shoulder level of the person; and
controlling, by at least one controller, the air shower gate to provide an air shower operation for the person in response to the at least one sensor's detection,
wherein the air shower gate comprises:
a frame installed between the indoor space of the building and the outdoor space,
the frame comprising a first vertical frame, a second vertical frame, and a top frame,
the first vertical frame extending in a vertical direction,
the second vertical frame extending in the vertical direction and being apart from the first vertical frame in a first horizontal direction,
the top frame extending in the first horizontal direction and connected to the first and second vertical frames,
the top frame extending between an indoor-side edge thereof and an outdoor-side edge thereof in a second horizontal direction perpendicular to the vertical direction and the first horizontal direction,
the top frame comprising an indoor-side air curtain outlet that extends in the first horizontal direction along the indoor-side edge thereof and is configured to generate an indoor-side air curtain that extends from the top frame to a floor in the vertical direction and also extends between the first and second vertical frames in the first horizontal direction,
the top frame comprising an outdoor-side air curtain outlet that extends in the first horizontal direction along the outdoor-side edge thereof and is apart from the indoor-side air curtain in the second horizontal direction, wherein the outdoor-side air curtain outlet is configured to generate an outdoor-side air curtain that extends from the top frame to the floor in the vertical direction, also extends between the first and second vertical frames in the first horizontal direction, and is apart from the indoor-side air curtain in the second horizontal direction,
wherein, when the top frame generates both the indoor-side air curtain and the outdoor-side air curtain, the air shower gate is configured to create an isolated air shower space defined by the frame, the indoor-side air curtain, and the outdoor-side air curtain,
the top frame further comprising a plurality of top air shower outlets configured to generate top air shower blows directed toward the floor, wherein the plurality of top air shower outlets are arranged along the first horizontal direction such that at least one of the plurality of top air shower outlets is located in a central portion of the top frame in the first horizontal direction,
the first vertical frame comprising a plurality of first side air shower outlets configured to generated first lateral air shower blows directed toward the second vertical frame, wherein the plurality of first side air shower outlets are arranged along the vertical direction such that at least one of the plurality of first side air shower outlets is located higher than others of the plurality of first side air shower outlets,
the second vertical frame comprising a plurality of second side air shower outlets configured to generated second lateral air shower blows directed toward the first vertical frame, wherein the plurality of second side air shower outlets are arranged along the vertical direction such that at least one the plurality of second side air shower outlets is located higher than others the plurality of second side air shower outlets,
at least one of the first vertical frame and the second vertical frame comprising at least one inlet configured to take air in,
wherein the air shower operation comprises:
in response to detecting the person in the entrance opening, generating the indoor-side air curtain and the outdoor-side air curtain to create the isolated air shower space separate from the indoor and outdoor spaces with the person staying inside the isolated air shower space, and
while maintaining the isolated air shower space separate from the indoor and outdoor spaces, generating the top air shower blows, the first lateral air shower blows and the second lateral air shower blows into the isolated air shower space to dust the person's body,
wherein, when generating the top air shower blows, based on the at least one sensor's detection of the person, the at least one controller inactivates at least one of the plurality of top air shower outlets located in the central portion of the top frame to avoid generating a top air blow that could be directed to the person's head from the top frame,
wherein, when generating the first lateral air shower blows, based on the at least one sensor's detection of the first person's shoulder level, the controller inactivates at least one of the plurality of first side air shower outlets to avoid generating a first lateral air blow that could be directed to the first person's head from the first vertical frame,
wherein, when generating the second air shower blows, based on the at least one sensor's detection of the first person's shoulder level, the controller inactivates at least one of the plurality of second side air shower outlets to avoid generating a second lateral air blow that could be directed to the first person's head from the second vertical frame,
wherein at least part of the first and second lateral air shower blows are generated in a direction inclined to the floor from the first and second horizontal directions which would avoid lifting of a skirt in case the first person wears the skirt.
